(19) United States
(12) Patent Application Publication
(10) Pub. No.: US 2021/0277202 A1
(43) Pub. Date: Sep. 9, 2021

(54) RECYCLING METHOD FOR STYRENE-CONTAINING PLASTIC WASTE

(71) Applicant: INEOS STYROLUTION GROUP GMBH, Frankfurt am Main (DE)

(72) Inventors: Bianca WILHELMUS, Hanau (DE); Norbert NIESSNER, Friedelsheim (DE); Hannes KERSCHBAUMER, Bad Soden am Taunus (DE); Mohammed ABBOUD, Riverside, IL (US)

(21) Appl. No.: 16/614,162

(22) PCT Filed: Jun. 5, 2018
(86) PCT No.: PCT/EP2018/064738

§ 371 Date: Nov. 15, 2019

Related U.S. Application Data
(60) Provisional application No. 62/515,690, filed on Jun. 6, 2017.

(51) Int. Cl. C08J 11/12; C08L 25/06; C07C 4/22; C10G 1/10; C10B 53/07

(52) U.S. Cl. CPC C08J 11/12; C08L 25/06; C07C 4/22; C10G 1/10; C10G 2300/1003; C08J 2325/06; C08J 2423/06; C08J 2423/12; C10B 53/07

(57) ABSTRACT

The invention relates to a method for economically using styrene-containing plastic waste as raw material for new high-quality plastic products as part of a raw material recycling process, optionally having the steps of pre-treating a styrene-containing starting material, decomposing the styrene-containing starting material in a suitable reactor, discharging and collecting the resulting gases and condensing the low-molecular products in a suitable separator, separating the collected low-molecular components of the previous step by means of a fractioning distillation process, and optionally additionally decomposing the styrene oligomers in a steam cracker.

The invention relates to a feedstock-recycling process for the cost-effective utilization of styrene-containing plastics wastes as feedstock for new high-quality plastics products.

The transition from a linear economy to a circular economy is necessary for both ecological and economic reasons in the light of climate change, environmental pollution, population growth and dependency on available resources. As early as the 1980s and 1990s, intensive efforts were made to develop processes for feedstock-recycling of plastics wastes, but because of unresolved process-technology problems and for economic reasons no industrial applications have appeared to date. Recycling of plastics wastes is generally divided into three types:

### a) Thermal Recycling

This type of recycling is currently the most important because the materials involved do not have to be of similar type. In the absence of any other recycling, the plastics wastes are incinerated, and it is therefore merely the energy liberated that is utilized. Although this approach saves equivalent quantities of petroleum, it does not meet the requirements of an economy that uses materials sustainably, and often produces environmentally harmful substances. In ecological terms, thermal recycling is therefore the least preferred recycling method.

### b) Materials Recycling

Materials recycling melts and repelletizes the used plastic so that it can be reprocessed to give semifinished and finished products. The problem here, however, is that if the pelletized material is not all of a single type the quality of the plastic becomes impaired (“downcycling”). This is in particular the case with “post-consumer” waste streams, which usually are mixtures of polymers. Complete separation into materials of the same type is very complicated, and is extremely difficult for multiphase plastics such as ABS. PET is an exception, with an existing bottle-collection system in which the waste collected intrinsically mostly comprises a single type of material. In the case of polystyrene, the predominant approach is in-factory recycling for industrial wastes. However, all forms of materials recycling cause thermal degradation of the polymers as a result of repeated melting, thus reducing molecular weight and polymer chain length, and impairing mechanical properties.

When polymers have suffered high exposure to heat and/or UV radiation over long periods, this being particularly relevant to plastics waste gathered from the environment, they have little suitability for this type of recycling simply because of said exposure.

### c) Feedstock Recycling:

Feedstock recycling of plastics wastes usually comprises cleaving of the macromolecules into individual parts, extending as far as monomers; these can be reused for synthesis of plastics directly or after purification, with no impairment of product quality by problematic contaminants or reduced chain lengths. The plastic is returned to the plastics cycle by way of the monomer. A specific form of feedstock recycling is use of plastics as reducing agents in blast furnaces, replacing the coke that is otherwise usually used. In Europe this route is scarcely practiced anywhere other than Austria, because a criticism that has to be leveled against the process is that it does not amount to recycling in the sense of reuse (closed-loop cycle).

A feedstock-recycling method for used plastics that can lead to the production of virgin polymers is the thermally induced depolymerization described in the literature (e.g. W. Kaminsky, Recycling of polymers by pyrolysis, in: Journal de Physique IV Colloque 03 (C7) (1993) C7-1543-C7-1552). In that document the polymer is cleaved thermally to give low-molecular-weight substances such as monomers and oligomers.

Pyrolysis (thermal decomposition) of plastics is known: DE 2310463 describes a process in which the depolymerization is carried out in a conventional twin-screw extruder. Introduction of large quantities of shear energy results in dissipative heating of the material. The decomposition temperature is thus exceeded, and the material decomposes. The volatile monomers are collected by way of appropriate devices, while the molten residue is discharged by extrusion from the extruder.

Another possibility is decomposition in a fluidized-bed reactor (W. Kaminsky, J. Menzel, H. Sinn, Recycling of plastics, in Conservation & Recycling, vol. 1 (1976) 91-110). A similar process is also described in EP 0649827. Plastics can also be decomposed in a microwave reactor, as described in WO 2015/024102. This process uses mixed household waste to produce a gas or an oil which can be used as fuel or for other steps in chemical synthesis. WO 2011/079894 describes the depolymerization of unsorted polymer mixtures in a reactor with introduction of thermal energy and with the aid of a catalyst.

A general disadvantage of use of catalysts in the pyrolysis of plastics waste, in particular of styrene-containing plastics waste, is that, because of the aromatic molecular structure and the large C/H ratio of styrene, a relatively large quantity of carbon is also formed and forms deposits on the catalyst surface, and rapidly renders the catalyst unusable.

In case of some polymer types, thermal decomposition does not lead to direct feedstocks for repolymerization: thermal decomposition of polyethylene, polypropylene and polyester merely gives non-specific products such as waxes, light oil and gases, which are not suitable for repolymerization; the only cost-effective possible use of these is as fuel. In contrast, polystyrene (PS) is one of the polymers that can be split into their monomers at relatively high temperatures, thus being suitable for repolymerization. Polystyrene, which is used in many sectors of everyday life, and also in single-use packaging, is therefore particularly suitable for the feedstock recycling desired here.

If polystyrene is heated substantially, decomposition products are formed. Products arising here are mainly styrene monomers, and also, in the case of incomplete decomposition, oligomers in the form of, for example, dimers, trimers and tetramers, and also, in the case of substantial decomposition, benzene, toluene, ethylbenzene and α-methylstyrene. The proportions of the individual constituents vary, and are substantially dependent on the experimental conditions, in particular on the temperature and the molecular weight of the polystyrene (C. Bouster, Study of the pyrolysis of polystyrenes: 1. Kinetics of thermal decomposition, in: Journal of Analytical and Applied Pyrolysis, 1 (1980) 297-313; C. Bouster, Evolution of the product yield with temperature and molecular weight in the pyrolysis of polystyrene, in: Journal of Analytical and Applied Pyrolysis 15 (1989) 249-259).

However, it is only the styrene monomers that can be used for repolymerization. Styrene oligomers are disruptive in repolymerization, because even small quantities of these are sufficient to influence essential properties of the polymer, and are therefore undesirable. The problem is that if the styrene monomers formed during decomposition of the polymer are not sufficiently rapidly cooled they react in a 4+2 Diels-Alder cycloaddition reaction to give oligomers; (see in this connection Kirchner, K. and Riederle, K., “Thermal polymerization of styrene—the formation of oligomers and intermediates, 1. Discontinuous polymerization up to high conversions”, in: Die Angewandte Makromolekulare Chemie 111 (1983) 1-16).

There are two possible sources for styrene oligomers: firstly incomplete decomposition of the starting polymer and secondly formation from styrene monomers after pyrolysis.

KR 2004-0088685 describes a method for reclaiming styrene from polystyrene-containing wastes via pyrolysis at 200 to 1000° C., but without any detailed discussion of the problem of oligomer formation.

U.S. Pat. No. 6,018,085 explains a method for obtaining styrene from polystyrene-containing materials contaminated with animal or vegetable fats, via thermal depolymerization of a solution of the materials in a solvent at 300-350° C., but with no reference to oligomer formation or methods of preventing same.

U.S. Pat. No. 9,650,313 discloses the thermal depolymerization, at 330-800° C., of polystyrene dissolved out of a polystyrene-containing waste. Here again, the problem of oligomer formation is not confronted.

U.S. Pat. No. 3,901,951 describes the pyrolysis of polystyrene, but provides no solution relating to minimization of oligomer formation or relating to utilization of oligomers.

Formation of the oligomers is in essence dependent on temperature and residence time in the reactor. The higher the temperature and the longer the residence time, the greater the quantity of undesired oligomers produced. However, temperature and residence time in the reactor cannot be reduced as desired, because there is a minimum temperature and a minimum residence time required to decompose the plastics waste. There is currently no method known to the person skilled in the art for complete prevention of oligomer formation.

Another matter that is not clear to the person skilled in the art, specifically because of the large number of possible processes, is that of conditions that can maximize the yield of styrene on an industrial scale. Table 1 lists some of the processes described, and yields reported, in the literature. For industrial scale, yields >95% are considered adequate.

TABLE 1
List of examples of some pyrolysis processes for the recycling
of polystyrene, and yields of said processes

| | | | Total yield of styrene |
|---|---|---|---|
| | | | monomer, styrene |
| | | Yield of styrene | dimer and styrene |
| Reference | Reaction conditions | monomer | trimer |
| C. Bouster, Evolution of | Flash pyrolysis at 500 to | 66.8% to 78.7%, | 70.3% to 86.4%, |
| the product yield with | 975° C. | dependent on reaction | dependent on reaction |
| temperature and | | temperature | temperature |
| molecular weight in the | | | |
| pyrolysis of polystyrene, | | | |
| Journal of Analytical | | | |
| and Applied Pyrolysis | | | |
| 15 (1989) 249-259 | | | |
| G. Audisio, Molecular | Pyrolysis at 600° C. | 79.5% to 90.7%, | 79.5% to 90.7%, |
| weight and pyrolysis | | dependent on molar | dependent on molar |
| products distribution of | | mass of polystyrene | mass of polystyrene |
| polymers. Polystyrene, | | used | used |
| in: Journal of Analytical | | | |
| and Applied Pyrolysis, | | | |
| 24(1) (1992)61-74 | | | |
| M. Bartoli, | Microwave-assisted | 36.7% to 50.1%, | 38.5% to 59.5%, |
| Depolymerization of | pyrolysis | dependent on reaction | dependent on reaction |
| polystyrene at reduced | | conditions | conditions |
| pressure through a | | | |
| microwave assisted | | | |
| pyrolysis, in: Journal of | | | |
| Analytical and Applied | | | |
| Pyrolysis, 113 (2015) | | | |
| 281-287 | | | |
| Y. S. Kim, Pyrolysis of | Pyrolysis at 370 to | 65.4% to 71.6%, | 87.5% to 94.6%, |
| polystyrene in a batch- | 400° C. | dependent on reaction | dependent on reaction |
| type stirred vessel. | | temperature | temperature |
| Korean Journal of | | | |
| Chemical Engineering, | | | |
| 16(2) (1999) 161-165 | | | |
| S. Ide, Controlled | Pyrolysis at 350 to | 35.4% to 64.5%, | 72.6% to 77.8%, |
| Degradation of | 500° C. | dependent on reaction | dependent on reaction |
| Polystyrene, in: J Appl | | temperature | temperature |
| Polym Sci, 29 (1984), | | | |
| 2561-2571. | | | |
| R. Lin, Acid-Catalyzed | Pyrolysis at 400° C. | 41% to 68%, dependent | 87% to 90%, dependent |
| Cracking of | | on molar mass of | on molar mass of |
| Polystyrene, J Appl | | polystyrene used | polystyrene used |
| Polym Sci, 63 (1997) | | | |
| 1287-1298. | | | |

Accordingly, there is a need for processes for the thermal decomposition of plastics waste which produce a maximal quantity of monomer, and also of other feedstocks that can be directly utilized in petrochemical processes, and which maximize separation of the styrene oligomers as byproduct and utilize these in another aspect of petrochemistry.

Examples of feedstocks that can be directly utilized in petrochemical processes are styrene, benzene, ethene and other naphtha constituents. The term styrene oligomers means styrene dimers, styrene trimers and other compounds that combine a plurality of styrene monomers.

It is an object of the invention to provide, for styrene-containing plastics wastes, a feedstock-recycling process which can use feedstocks made up of various plastics and which produces a large quantity of styrene monomer alongside other feedstocks that can be utilized in petrochemical processes.

The object is achieved via a process for the pyrolytic depolymerization of a styrene-containing plastics waste (K) comprising or consisting of the components A, B1, B2, B3 and C:
A: 0.1 to 100% by weight, based on the entirety of components A, B1, B2 and B3, of at least one styrene-containing polymer comprising at least 40% of styrene, preferably at least 50% by weight of styrene, particularly preferably at least 80% by weight of styrene, in particular at least 85% by weight of styrene, based in each case on component A, and up to 60% by weight, based on component A, of rubber and/or other comonomers which do not interrupt the pyrolysis process, in particular acrylonitrile, vinyl chloride, methyl methacrylate and/or alpha-methylstyrene, preferably less than 20% by weight of acrylonitrile, vinyl chloride, methyl methacrylate and/or alpha-methylstyrene, particularly preferably less than 10% by weight of acrylonitrile, vinyl chloride, methyl methacrylate and/or alpha-methylstyrene;
B1: 0 to 60% by weight, based on the entirety of A, B1, B2 and B3, of polyolefin or polyolefin mixtures, for example polyethylene or polypropylene;
B2: 0 to 60% by weight, based on the entirety of A, B1, B2 and B3, of other polymers, differing from A and B1, for example polycarbonates, polyesters, polyamides and/or polyvinyl chloride;
B3: 0 to 20% by weight, based on the entirety of A, B1, B2 and B3, of conventional plastics additives and conventional plastics auxiliaries;
C: 0 to 50% by weight, based on the entirety of A, B1, B2 and B3, of other foreign substances, dirt and moisture;

Comprising (or consisting of) the following steps:
i) decomposition of the styrene-containing plastics waste (K) in a suitable reactor via introduction of thermal energy and optionally of shear energy, where the plastics waste (K) to be decomposed is introduced into a pyrolysis zone of the reactor and is pyrolyzed there at a temperature of 200° C. to 800° C., preferably 250° C. to 500° C. (average temperature of the reaction mixture measured at the inner surface of a reactor wall (during the reaction time)), where the residence time in the pyrolysis zone of the plastics waste (K) to be pyrolyzed is 0.1 to 60 minutes, and where the styrene component (A) of the styrene-containing plastics waste (K) is at least to some extent decomposed to give styrene monomers and styrene oligomers;
ii) discharge and collection of the gases produced in step i) and condensation of the low-molecular-weight products comprising the resultant styrene monomers, in a suitable separator;
iii) fractionation, by means of fractional distillation, of the condensed low-molecular-weight constituents of the previous step comprising the resultant styrene monomers and
iv) optionally introduction of the styrene oligomers formed in step i), and also any styrene oligomers present before step i), into a steam cracker.

The process of the invention provides a high yield of styrene monomers and permits further utilization of the styrene oligomer fraction in petrochemistry.

### Styrene-Containing Plastics Waste (K)

Suitable starting materials for the process of the invention are styrene-containing plastics wastes (K) comprising (and preferably consisting of) components A, B1, B2, B3 and C:

A: styrene-containing polymer with at least 40% by weight of styrene, preferably at least 50% by weight of styrene, particularly preferably at least 80% by weight of styrene, in particular at least 85% by weight of styrene.

Other constituents of component A are optionally up to 60% by weight, preferably 50% by weight, based on component A, of rubber and/or other comonomers which do not interrupt the pyrolysis process, in particular acrylonitrile, vinyl chloride, methyl methacrylate or alpha-methylstyrene. Preference is given to less than 20% by weight of acrylonitrile, vinyl chloride, methyl methacrylate or alpha-methylstyrene, and particular preference is given to less than 10% by weight of acrylonitrile, vinyl chloride, methyl methacrylate or alpha-methylstyrene.

The quantity of component A in the styrene-containing plastics waste (K) is 0.1 to 100% by weight, preferably at least 1.0% by weight, particularly preferably at least 10% by weight, in particular at least 50% by weight, based in each case on the entirety of components A, B1, B2 and B3.

B1: 0 to 60% by weight, based on the entirety of A, B1, B2 and B3, of polyolefins and polyolefin mixtures, for example polyethylene, polypropylene.

B2: 0 to 60% by weight, based on the entirety of A, B1, B2 and B3, of other polymers differing from A and B1, for example polycarbonates, polyesters, polyamides and/or polyvinyl chloride.

B3: 0 to 20% by weight, based on the entirety of A, B1, B2, and B3, of conventional plastics additives and conventional plastics auxiliaries. By way of example, an additive or an auxiliary can be selected from the group consisting of antioxidants, UV stabilizers, peroxide destroyers, antistatic agents, lubricants, mold-release agents, flame retardants, fillers and reinforcing materials (glass fibers, carbon fibers, etc.), colorants and combinations of two or more thereof.

C: 0 to 50% by weight, based on the entirety of A, B1, B2 and B3, of other foreign substances, dirt and moisture. Where appropriate, the starting material is to be pretreated as described above by washing processes until it comprises no more than 50% by weight, preferably no more than 30% by weight and particularly preferably no more 20% by weight, of other foreign substances, dirt and moisture, based on the entirety of A, B1, B2 and B3.

Where appropriate, in particular when the proportion of C) is relatively high, the styrene-containing plastics waste (K) used in the invention is pretreated in a suitable manner in order to remove other substances or compositions, for example adhering contaminants such as food residues or dirt, moisture and foreign substances such as metals or other substances and composite materials.

This is advantageously achieved in a pretreatment which can comprise one or more of the following steps, where the sequence of the steps is not fixed and multiple repetition of steps is also possible: manual sorting to remove disruptive substances, washing, comminution, automatic sorting in suitable systems. Where appropriate, styrene-containing plastics wastes not falling within the specification of plastics wastes (K) can also be converted by falling within such a process into materials (K) used in the invention, the aim here being to obtain a styrene-containing plastics waste complying with the specifications of (K).

Preference is therefore also given to an embodiment of the process of the invention in which a styrene-containing plastics waste (K) is subjected in a step o) to a pretreatment which comprises one or more of the following steps, where the sequence of the steps is not fixed and multiple repetition of steps is also possible: manual sorting to remove disruptive substances, washing, comminution, automatic sorting in suitable systems, the aim here being to obtain the styrene-containing plastics waste (K) of the invention.

Component A preferably comprises:

Styrene-containing polymer A with A1: at least 40% by weight of styrene, preferably at least 50% by weight of styrene, particularly preferably at least 80% by weight of styrene, in particular at least 85% by weight of styrene, and A2: other comonomers, for example butadiene, (meth)acrylate, acrylonitrile, alpha-methylstyrene, phenylmaleimide.

Component A is moreover preferably selected from the class of the non-impact-resistant or impact-modified polystyrenes consisting of the vinylaromatic copolymers selected from the group consisting of: styrene-acrylonitrile copolymers, α-methylstyrene-acrylonitrile copolymers, styrene-maleic anhydride copolymers, styrene-phenylmaleimide copolymers, styrene-methyl methacrylate copolymers, styrene-acrylonitrile-maleic anhydride copolymers, styrene-acrylonitrile-phenylmaleimide copolymers, α-methylstyrene-acrylonitrile-methyl methacrylate copolymers, α-methylstyrene-acrylonitrile-tert-butyl methacrylate copolymers and styrene-acrylonitrile-tert-butyl methacrylate copolymers.

Impact modifiers A3 can also optionally be present.

These consist by way of example of A31: 20-90% by weight of a graft base of one or more monomers consisting of:
A311: 70 to 100% by weight of at least one conjugated diene and/or at least one acrylate;
A312: 0 to 30% by weight of at least one other comonomer selected from: styrene, α-methylstyrene, acrylonitrile, methacrylonitrile, MMA, MA and N-phenylmaleimide (N-PMI);
A313: 0 to 10% by weight of one or more polyfunctional crosslinking monomers which, if component A211 is acrylate, is present in quantities of at least 0.1% by weight,
A32: 10 to 80% by weight of a graft of a monomer or of a plurality of monomers consisting of:
A321: 65 to 100% by weight, preferably 70 to 100% by weight, particularly preferably 75 to 95% by weight, of at least one vinylaromatic monomer, preferably styrene and/or α-methylstyrene, in particular styrene;
A322: 5 to 35% by weight, preferably 10 to 30% by weight, particularly preferably 15 to 25% by weight, of acrylonitrile and/or methacrylonitrile, preferably acrylonitrile,
A323: 0 to 30% by weight, preferably 0 to 20% by weight, particularly preferably 0 to 15% by weight, of at least one other monoethylenically unsaturated monomer selected from: MMA, MA and N-PMI;
   where the entirety of A31 and A32 provides 100% by weight, based on A3, and
   where the proportion of comonomers A2 in the entirety of component A is no higher than 30% by weight, preferably no higher than 15% by weight, particularly preferably no higher than 8% by weight and in particular no higher than 5% by weight.

The following are preferred as polymer A: polystyrene (unmodified standard polystyrene and/or impact-modified polystyrene), styrene-acrylonitrile copolymers (SAN), styrene-methyl methacrylate copolymers (SMMA) and/or styrene-maleic anhydride copolymers (SMA). Particular preference is given to styrene-acrylonitrile copolymers.

SAN copolymers and α-methylstyrene-acrylonitrile copolymers (AMSAN) used as polymers A of the invention generally comprise 18 to 35% by weight, preferably 20 to 32% by weight, particularly preferably 22 to 30% by weight, of acrylonitrile (AN) and 82 to 65% by weight, preferably 80 to 68% by weight, particularly preferably 78 to 70% by weight, of styrene (S) and, respectively, α-methylstyrene (AMS), where the entirety of styrene and, respectively, α-methylstyrene and acrylonitrile provides 100% by weight. The average molar mass Mw of the SAN and AMSAN copolymers is generally 50 000 to 500 000 g/mol, preferably 100 000 to 350 000 g/mol, particularly preferably 100 000 to 300 000 g/mol, and very particularly preferably 150 000 to 250 000 g/mol.

SMMA copolymers used as polymer A in the invention generally comprise 18 to 50% by weight, preferably 20 to 30% by weight, of methyl methacrylate (MMA) and 50 to 82% by weight, preferably 80 to 70% by weight, of styrene, where the entirety of styrene and MMA provides 100% by weight.

SMA copolymers used as polymer A in the invention generally comprise 10 to 40% by weight, preferably 20 to 30% by weight, of maleic anhydride (MA) and 60 to 90% by weight, preferably 80 to 70% by weight, of styrene, where the entirety of styrene and MA provides 100% by weight.

The following can be used as conjugated dienes: dienes having 4 to 8 carbon atoms, for example butadiene, isoprene, piperylene and chloroprene and mixtures of these. It is preferable to use butadiene or isoprene or a mixture of these; it is very particularly preferable to use butadiene.

Diene rubbers are by way of example homopolymers of the abovementioned conjugated dienes, copolymers of said dienes with one another, copolymers of said dienes with acrylates, in particular n-butyl acrylate, and copolymers of said dienes with comonomers selected from styrene, α-methylstyrene, acrylonitrile, methacrylonitrile, methyl methacrylate (MMA), maleic anhydride (MA) and N-phenylmaleimide (N-PMI). The diene rubbers can also comprise polyfunctional monomers having crosslinking action, as mentioned above for the acrylate rubbers.

### Component B1

The following can be used by way of example as component B1: polyolefins such as LDPE (low-density polyethylene), LLDPE (linear low-density polyethylene), HDPE (high-density polyethylene), metallocene polyethylenes, ethylene copolymers such as poly(ethylene-co-vinyl acetate), ethylene-butene, ethylene-hexene, ethylene-octene copolymers, and also cycloolefin copolymers, individually or in the form of mixture. The following can also be used by way of example as component B1: polypropylene, for example homo- or copolymers of propylene, metallocene-catalyzed polypropylenes, and also copolymers of propylene with other comonomers known to the person skilled in the art.

The proportion of component B present in the styrene-containing plastics waste (K) used in the invention is 0 to 60% by weight, based on the entirety of components A, B1, B2 and B3; in a preferred embodiment it is 0.1 to 50% by weight, based on the entirety of components A, B1, B2 and B3.

### Component B2

The styrene-containing plastics waste (K) used in the invention can additionally comprise 0 to 60% by weight, based on the entirety of A, B1, B2, B3, of at least one other polymer B2 differing from the polymers A and B1, for example selected from polycarbonates, polyamides, poly(meth)acrylates, polyvinyl chlorides, polyester, halogenated polymers, vinylaromatic-diene copolymers (SBC), polyethers, polysulfones, polyether sulfones, polyimidazoles and related polymers.

### Component B3

The following can be present as component B3: 0 to 20% by weight, based on the entirety of A, B1, B2, B3, of conventional plastics additives and conventional plastics auxiliaries. By way of example, an additive or an auxiliary can be selected from the group consisting of antioxidants, UV stabilizers, peroxide destroyers, antistatic agents, lubricants, mold-release agents, flame retardants, fillers and reinforcing materials (glass fibers, carbon fibers, etc.), colorants and combinations of two or more thereof. The following may be mentioned as examples of oxidation retarders and heat stabilizers: halides of metals of group I of the periodic table of the elements, e.g. sodium halides, potassium halides and/or lithium halides, optionally in conjunction with copper(I) halides, e.g. chlorides, bromides, iodides, sterically hindered phenols, hydroquinones, various substituted members of these groups and mixtures of these in concentrations up to 1% by weight, based on the weight of the entirety of A, B1, B2 and B3.

The following may be mentioned as UV stabilizers, which are generally present in quantities up to 2% by weight, based on the entirety of A, B1, B2 and B3: various substituted resorcinols, salicylates, benzotriazoles and benzophenones.

The styrene-containing plastics waste (K) used in the invention can moreover comprise, as colorants, organic dyes such as nigrosin, pigments such as titanium dioxide, phthalocyanines, ultramarine blue and carbon black, and also fibrous and pulverulent fillers and reinforcing agents. Examples of the latter are carbon fibers, glass fibers, amorphous silica, calcium silicate (wollastonite), aluminum silicate, magnesium carbonate, kaolin, chalk, powdered quartz, mica and feldspar. The proportion of these fillers and colorants is generally up to 50% by weight, preferably up to 35% by weight.

The following can be present by way of example as nucleating agents: talc, calcium fluoride, sodium phenylphosphinate, aluminum oxide, silicon dioxide and nylon 22.

Examples of lubricants and mold-release agents, quantities used of which are generally up to 1% by weight, are long-chain fatty acids such as stearic acid or behenic acid, salts thereof (e.g. Ca stearate or Zn stearate) and esters (e.g. stearyl stearate or pentaerythritol tetrastearate), and also amide derivatives (e.g. ethylenebisstearylamide).

Quantities of up to 0.1% by weight of mineral-based antiblocking agents can moreover be present. The following may be mentioned as examples: amorphous or crystalline silica, calcium carbonate and aluminum silicate.

Quantities of up to 5% by weight, preferably up to 2% by weight, of mineral oil, preferably medicinal white oil, can be present as processing aid.

The following may be mentioned as examples of plasticizers: dioctyl phthalate, dibenzyl phthalate, butyl benzyl phthalate, hydrocarbon oils, N-(n-butyl)benzenesulfonamide and o- and p-tolylethylsulfonamide.

Any of the flame retardants known for the respective thermoplastics can moreover be present, in particular phosphorus itself or those based on phosphorus compounds.

### Step i

Step (i) of the process of the invention involves decomposition of the styrene-containing plastics waste (K) in a suitable pyrolysis reactor via introduction of thermal energy and optionally of shear energy. For this, the material to be decomposed is introduced into a pyrolysis zone of the reactor and is pyrolyzed there at a temperature of 200° C. to 800° C., preferably 250° C. to 500° C. ((average) temperature of the reaction mixture measured at the inner surface of a reactor wall during the reaction time), where the residence time in the pyrolysis zone of the material to be pyrolyzed is 0.1 to 60 minutes. Particular preference is given to pyrolysis temperatures of 280° C. to 470° C. and to residence times of 1 to 45 minutes; particular preference is given to pyrolysis temperatures of 300° C. to 450° C. and to residence times of 2 to 30 minutes.

The following are suitable by way of example as pyrolysis reactors: the abovementioned twin-screw extruders, fluidized-bed reactors and microwave reactors.

Introduction of thermal energy can be achieved by way of example by heating or by microwave irradiation.

It is preferable that the pyrolysis reactor used in the invention comprises no catalyst.

In another preferred embodiment, shear energy is additionally introduced, alongside thermal energy, into the styrene-containing plastics waste (K).

### Steps ii and iii

Step ii) involves the discharge and collection, in a suitable separator, of the gases and condensation of the low-molecular-weight products arising in step i) and comprising the styrene monomers formed in step i). Step iii) includes the fractionation of the collected low-molecular-weight constituents of the previous step by means of fractional distillation. The styrene monomers isolated are then available for repolymerization. Suitable devices for the steps ii) and iii) are known and familiar to the person skilled in the art.

### Step iv)

Finally, the (optional) step iv) of the process of the invention consists in the introduction of the styrene oligomers formed in step i), and also any styrene oligomers present before step i), into a steam cracker in which the oligomers undergo further cracking, so that this material, too, can yield starting materials, for example ethene, propene or benzene, for plastics.

Steam cracking is a thermal decomposition process which is carried out in the presence of steam. It is the most important industrial process for the production of fundamental chemical substances such as ethene and propene from petroleum. Starting materials are typically saturated hydrocarbons such as ethane, propane, butanes or naphtha. The hydrocarbons are mixed with steam in the cracking process and heated in a cracking furnace to temperatures of typically 800 to 850° C. The residence time in the tube coils of the directly fired furnace is between 0.1 and 0.5 seconds.

The steam serves firstly to reduce formation of coke on the internal walls of the tube, but also serves to shift the equilibrium of the thermal reaction toward the target products (inter alia ethene and propene). The short residence time in the reaction zone and the subsequent rapid cooling of the cracking gas serve to increase selectivity, which is defined for steam cracking via the ratio of methane to propene.

The combination of the separation processes mentioned in steps ii) and iii) with steam cracking in step iv) permits very high utilization of all of the volatile cracking products of the plastics waste.

According to the invention, “styrene-containing” and “% by weight of styrene” in connection with styrene polymers always refer to styrene incorporated within a polymer.

### EXPLANATION OF THE DRAWINGS

FIG. 1 shows an example of a GC-MS analysis of the reaction products from example 1.

FIG. 2 shows an example of a GC analysis of the reaction products from example 2.

The invention is illustrated by the examples, figures and claims below.

### EXAMPLES

In order to demonstrate the suitability of styrene-containing plastics wastes, polymers are heated in flasks to 350° C. to 450° C. This temperature is the average temperature of the reaction mixture in the interior of the reaction vessel during the reaction time.

TABLE 2
Inventive examples and comparative examples

| | Inv. Ex. 1 | Inv. Ex. 2 | V1 | V2 | V3 | V4 | Inv. Ex. 3 | Inv. Ex. 4 |
|---|---|---|---|---|---|---|---|---|
| (K) | PS GPPS | PS 468N | PP | PP Co- | LDPE | LLDPE | Mixture of | Mixture of |
| | 158 N | (impact- | Homo- | polymer | | | 60% GPPS | 60% GPPS |
| | | modified | polymer | | | | 158 N | 158 N |
| | | poly- | | | | | polystyrene | polystyrene |
| | | styrene) | | | | | and 40% PP | and 40% |
| | | | | | | | homo- | LDPE |
| | | | | | | | polymer | |
| Main | Styrene | Styrene | Waxy | Waxy | Waxy | Waxy | Styrene | Styrene |
| products of | monomer | monomer | sub- | sub- | sub- | sub- | monomer | monomer |
| thermal | and | and | stances | stances | stances | stances | and styrene | and styrene |
| cracking | styrene | styrene | | | | | oligomers | oligomers |
| | oligomers | oligomers | | | | | | |

V = comparative examples

### Inventive Example 1

The polymer samples are decomposed in a glass apparatus consisting of round-bottomed flask with heating jacket, Liebig condenser and cold trap. The commercially available polystyrene (PS GPPS 158 N, producer: INEOS Styrolution, Frankfurt) for decomposition is charged to the round-bottomed flask, input weight being 100 g. A vacuum pump is used to generate subatmospheric pressure in the apparatus. The residual pressure in the apparatus is 45 mbar. The start temperature of the reaction is 370° C., measured between heating jacket and flask.

Formation of condensate starts at a jacket temperature of 460° C. At a jacket temperature of 550° C. the reaction has concluded. The average reaction temperature is 460° C. During the entire running time of the experiment, the reaction products are collected in two stages, firstly in a cryostat at −40° C. and then in a cold trap cooled to −196° C. by liquid nitrogen. The yield of condensate is 95.4%, based on the quantity of polystyrene used.

The reaction products produced are characterized by means of GC-MS

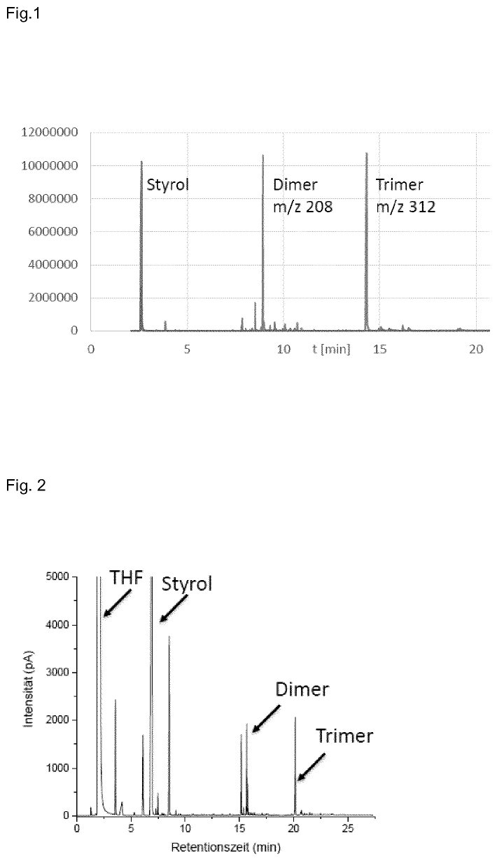

(Agilent 7890A gas chromatograph, Agilent DB-1 column with He carrier gas), see FIG. 1. Table 3 describes the composition of the reaction products.

TABLE 3
Composition of reaction products from inventive example 1

| | Proportion in | Proportion based on |
|---|---|---|
| Component | condensate | starting material |
| Styrene monomer | 37.25% | 35.63% |
| Styrene dimer | 19.73% | 18.82% |
| Styrene trimer | 32.37% | 30.88% |
| Total proportion of styrene | 89.35% | 85.23% |
| and styrene oligomers | | |

In the next step, the condensate is fractionated by fractional distillation. This is achieved by distillation with a Vigreux column at subatmospheric pressure generated by a membrane pump (starting pressure 50 mbar). A distillation pig is used to collect the various fractions, and the low-boiling-point fraction is collected in a cold trap cooled by liquid nitrogen. The distillation temperature here is increased from room temperature to 490° C. (flask jacket temperature). The resultant fractions are characterized by gas chromatography (see table 4). The expression “low boilers” here means all of the substances evolved from the condensate to be fractionated that change to the gas phase at a lower temperature than styrene monomer, styrene dimer and styrene trimer. The expression “high boilers” here means all of the substances evolved from the condensate to be fractionated that change to the gas phase at a higher temperature than styrene monomer, styrene dimer and styrene trimer.

TABLE 4
Characterization of the fractions from inventive example 1

| | | | | | | | Total | |
|---|---|---|---|---|---|---|---|---|
| | | | | | | | propor- | |
| | Temper- | | | | | | tion of | Propor- |
| | ature | Propor- | Propor- | Propor- | Propor- | Propor- | styrene and | tion of |
| | of heating | tion of | tion of | tion of | tion of | tion of | styrene | fraction, |
| | medium | low | high | styrene | styrene | styrene | oligomers | based on |
| Fraction | (° C.) | boilers | boilers | monomer | dimer | trimer | in fraction | condensate |
| 1 | 82 | 8.94% | — | 91.06% | — | — | 91.06% | 0.80% |
| 2 | 86 | 1.17% | — | 98.83% | — | — | 98.83% | 28.03% |
| 3 | 88 | — | — | 100.00% | — | — | 100.00% | 10.80% |
| 4 | 95 | — | — | 100.00% | — | — | 100.00% | 5.20% |
| 5 | 116 | — | — | 100.00% | — | — | 100.00% | 2.23% |
| 6 | 125 | — | 0.42% | 99.58% | — | — | 99.58% | 1.88% |
| 7 | 133 | — | 0.71% | 99.29% | — | — | 99.29% | 0.48% |
| 8 | 141 | — | 1.56% | 98.44% | — | — | 98.44% | 1.18% |
| 9 | 451 | 4.66% | 21.12% | 39.57% | 31.88% | 2.76% | 74.21% | 0.50% |
| 10 | 453 | — | 11.71% | 23.07% | 64.20% | 1.02% | 88.29% | 0.50% |
| 11 | 453 | — | 7.76% | 8.61% | 60.84% | 22.79% | 92.24% | 2.83% |
| 12 | 490 | — | 8.67% | 8.86% | 79.63% | 2.83% | 91.06% | 12.83% |
| Cold | | 0.96% | 4.83% | 86.74% | 6.85% | 0.61% | 91.32% | 0.63% |
| trap | | | | | | | | |
| Residue | | | | | | | | 32.15% |
| in flask | | | | | | | | |

### Inventive Example 2

The experiment is repeated by analogy with inventive example 1, using impact-modified polystyrene (PS 486N, producer: INEOS Styrolution). PS 486N polystyrene is an impact-resistant amorphous polystyrene (HIPS) with melt volume flow rate (melt volume rate 200° C./5 kg load, ISO 1133) about 4 cm³/10 min.

The starting temperature of the reaction is 370° C., measured between heating jacket and flask. Formation of condensate starts at a jacket temperature of 450° C. At a jacket temperature of 550° C. the reaction has concluded. During the entire running time of the experiment, the reaction products are collected in two stages, firstly in a cryostat at −40° C. and then in a cold trap cooled to −196° C. by liquid nitrogen. The yield of condensate is 82.2%, based on the quantity of polystyrene used. The reaction products produced are characterized by means of gas chromatography (Agilent 7890A gas chromatograph, Agilent HP-5 column with argon carrier gas, detection by flame ionization, solvent THF), see FIG. 2.

Table 5 describes the composition of the reaction products.

TABLE 5
Composition of reaction products from inventive example 2

| | Proportion in | Proportion based on |
|---|---|---|
| Component | condensate | starting material |
| Styrene monomer | 52.59% | 43.23% |
| Styrene dimer | 6.50% | 5.34% |
| Styrene trimer | 10.48% | 8.61% |
| Total proportion of styrene | 69.57% | 57.18% |
| and styrene oligomers | | |

In the next step, the condensate is fractionated by fractional distillation. This is achieved by distillation with a Vigreux column at subatmospheric pressure generated by a membrane pump. A distillation pig is used to collect the various fractions, and the low-boiling-point fraction is collected in a cold trap cooled by liquid nitrogen. The distillation temperature here is increased from room temperature to 160° C. (flask jacket temperature). The resultant fractions are characterized by gas chromatography (see table 6). The expression “low boilers” here means all of the substances evolved from the condensate to be fractionated that change to the gas phase at a lower temperature than styrene monomer, styrene dimer and styrene trimer. The expression “high boilers” here means all of the substances evolved from the condensate to be fractionated that change to the gas phase at a higher temperature than styrene monomer, styrene dimer and styrene trimer. Table 6 collates styrene monomer, styrene dimer and styrene dimer within a single column.

TABLE 6
Characterization of fractions from inventive example 2

| | Temper- | | | | Propor- |
|---|---|---|---|---|---|
| | ature | Propor- | Propor- | Propor- | tion of |
| | of heating | tion of | tion of | tion of | fraction |
| | medium | low | high | styrene | based on |
| Fraction | (° C.) | boilers | boilers | monomer | condensate |
| 1 | 78 | 48.88% | 2.34% | 48.78% | 3.35% |
| 2 | 80 | 41.97% | 4.05% | 55.68% | 2.60% |
| 3 | 100 | 15.80% | 7.09% | 77.11% | 39.55% |
| 4 | 118 | 5.95% | 8.94% | 85.11% | 5.45% |
| 5 | 130 | 2.42% | 7.73% | 89.85% | 0.45% |
| 6 | 145 | 1.35% | 8.33% | 90.32% | 0.53% |
| 7 | 149 | 0.80% | 15.16% | 84.04% | 0.13% |
| 8 | 160 | 2.53% | 17.90% | 79.57% | 0.25% |
| Cold trap | | 60.94% | 1.23% | 37.83% | 1.23% |
| Residue | | | | | 46.48% |
| in flask | | | | | |

Analogous experiments are carried out with a polymer blend made of 85% by weight of polystyrene (A), 8% by weight of aromatic polycarbonate (B2), 2% by weight of conventional additives (B3) and 5% of foreign substances (C).

1-13. (canceled)

14. A process for the pyrolytic depolymerization of a styrene-containing plastics waste (K) consisting of the components A, B1, B2, B3 and C:
A: 0.1 to 100% by weight, based on the entirety of components A, B1, B2 and B3, of at least one styrene-containing polymer comprising at least 40% of styrene, based in each case on component A, and up to 60% by weight, based on component A, of rubber and/or other comonomers which do not interrupt the pyrolysis process;
B1: 0 to 60% by weight, based on the entirety of A, B1, B2 and B3, of polyolefin or polyolefin mixtures;
B2: 0 to 60% by weight, based on the entirety of A, B1, B2 and B3, of other polymers, differing from A and B1;
B3: 0 to 20% by weight, based on the entirety of A, B1, B2 and B3, of conventional plastics additives and conventional plastics auxiliaries;
C: 0 to 50% by weight, based on the entirety of A, B1, B2 and B3, of other foreign substances, dirt and moisture;
the process comprising the steps of:
i) decomposing the styrene-containing plastics waste (K) in a suitable reactor via introduction of thermal energy and optionally of shear energy, where the plastics waste (K) to be decomposed is introduced into a pyrolysis zone of the reactor and is pyrolyzed there at an average temperature of 250° C. to 500° C. measured at the inner surface of a reactor wall during the reaction time, where the residence time in the pyrolysis zone of the plastics waste (K) to be pyrolyzed is 0.1 to 60 minutes, and where the styrene component of the styrene-containing plastics waste (K) is at least to some extent decomposed to give styrene monomers and styrene oligomers;
ii) discharging and collecting the gases produced in step i) and condensing the low-molecular-weight products comprising the resultant styrene monomers, in a suitable separator;
iii) fractionating, by means of fractional distillation, the condensed low-molecular-weight constituents of the previous step comprising the resultant styrene monomers.

15. The process as claimed in claim 14, characterized in that in a further step iv) the resultant styrene oligomers from step i), and also any styrene oligomers present before step i), are introduced into a steam cracker.

16. The process as claimed in claim 14, characterized in that the quantity present of component A, based on the entirety of components A, B1, B2 and B3, is at least 1% by weight.

17. The process as claimed in claim 14, characterized in that the quantity present of component A, based on the entirety of components A, B1, B2 and B3, is at least 10% by weight.

18. The process as claimed in claim 14, where component A comprises 0 to 10% by weight, based on component A, of one or more comonomers from the group consisting of acrylonitrile, vinyl chloride, methyl methacrylate and alpha-methylstyrene.

19. The process as claimed in claim 14, where the proportion of component B1 is 0.1 to 50% by weight, based on the entirety of components A, B1, B2 and B3.

20. The process as claimed in claim 14, where the proportion of component C is 0.1 to 30% by weight based on the entirety of components A, B1, B2 and B3.

21. The process as claimed in claim 14, where the quantity of component A present is at least 50% by weight, based on the entirety of components A, B1, B2 and B3, where the quantity of component B1 present is at least 10% by weight, based on the entirety of components A, B1, B2 and B3, and the proportion of component C is 0.1 to 20% by weight, based on the entirety of components A, B1, B2 and B3.

22. The process as claimed in claim 14, where the pyrolysis temperature in step i) is in the range 280 to 470° C. and the residence time in step i) is in the range 1 to 45 minutes.

23. The process as claimed in claim 14, where the pyrolysis reactor in step i) is selected from the group consisting of twin-screw extruders, fluidized-bed reactors and microwave reactors.

24. The process as claimed in claim 14, where shear energy is additionally introduced in step i) into the styrene-containing plastics waste (K).

25. The process as claimed in claim 14, where the pyrolysis reactor in step i) comprises no catalyst.

26. The process as claimed in claim 14, where the styrene-containing plastics waste (K) is subjected in a preceding step o) to a pretreatment comprising one or more of the following steps, where the sequence of the steps is not fixed and multiple repetition of steps is also possible: manual sorting to remove disruptive substances, washing, comminution, automatic sorting in suitable systems.

27. The process as claimed in claim 14, where
component A comprises at least one styrene-containing polymer comprising at least 50% of styrene;
component B1 is polyolefin or polyolefin mixtures selected from polyethylene, polypropylene and mixtures containing them;
component B2 is a polymer, differing from A and B1, selected from polycarbonates, polyesters, polyamides, polyvinyl chloride, and mistuxtures thereof.

28. The process as claimed in claim 22, where the pyrolysis temperature in step i) is in the range 300 to 450° C. and the residence time in step i) is in the range 2 to 30 minutes.
